(19) United States
(12) Patent Application Publication
(10) Pub. No.: US 2013/0158415 A1
(43) Pub. Date: Jun. 20, 2013

(54) BALLISTOCARDIOGRAM ANALYSIS APPARATUS AND METHOD, AND SYSTEM FOR UTILIZING BALLISTOCARDIOGRAM FOR VEHICLE USING THE SAME

(71) Applicants: Gil Ju KIM, Hwaseong-si (KR); Sun Woo CHOI, Gwangmyeong-si (KR); Sang Man SEO, Ansan-si (KR); In Ho LEE, Hwaseong-si (KR); Tae Hyoung YANG, Seoul (KR); Hee Chan KIM, Seoul (KR); Seung Woo NOH, Seoul (KR); Chi Yul YOON, Seoul (KR)

(72) Inventors: Gil Ju KIM, Hwaseong-si (KR); Sun Woo CHOI, Gwangmyeong-si (KR); Sang Man SEO, Ansan-si (KR); In Ho LEE, Hwaseong-si (KR); Tae Hyoung YANG, Seoul (KR); Hee Chan KIM, Seoul (KR); Seung Woo NOH, Seoul (KR); Chi Yul YOON, Seoul (KR)

(73) Assignees: Hyundai Motor Company, Seoul (KR); SNU R&DB Foundation, Seoul (KR); Kia Motors Corporation, Seoul (KR)

(21) Appl. No.: 13/533,803

(22) Filed: Jun. 26, 2012

(30) Foreign Application Priority Data
Dec. 15, 2011 (KR) 10-2011-0135569

(51) Int. Cl. A61B 5/0205; A61B 5/11

(52) U.S. Cl. 600/483

(57) ABSTRACT

A ballistocardiogram (BCG) analysis apparatus, method, and system is provided for utilizing the BCG for a vehicle. A basic pattern is selected when measurement of a BCG is initiated. Data about BCG is measured for a predetermined period of time. Whether the basic pattern is suitable for the measured data is determined by comparing the measured data with the basic pattern. If the basic pattern is suitable for the measured BCG data, continuous data about the BCG is collected. Signal processing is performed based on pattern matching using the continuous data. A biological condition of the examinee is recognized using results of the signal processing. A posture of the examinee is measured, and if the posture of the examinee has changed, a pattern DB is searched, the basic pattern is changed to a suitable pattern, and then signal processing is performed.

### CROSS-REFERENCE TO RELATED APPLICATIONS

The present application claims priority of Korean Patent Application Number 10-2011-0135569 filed Dec. 15, 2011, the entire contents of which application is incorporated herein for all purposes by this reference.

### BACKGROUND OF INVENTION

1. Field of Invention

The present invention relates to a technology for measuring and analyzing the ballistocardiogram of a passenger, and performing follow-up actions, such as performing ventilation or massaging the passenger, using the ballistocardiogram.

2. Description of Related Art

A ballistocardiogram (BCG) refers to a measure of a reaction force on a body to the ejection of blood from the heart, and then enables the condition of the heart to be estimated. The principle of measurement is to obtain a BCG signal by measuring a minute variation in the acceleration of blood or a variation in the weight of a body depending on the reaction to the ejection of blood from the heart.

FIG. 1 illustrates the principle of the conventional measurement of a BCG. That is, a sensor capable of measuring the pressure or weight is disposed between a human being and the ground and is configured to measure variations over time. As shown in the drawing, a BCG is measured together with an Electrocardiogram (ECG) and is then used to determine the magnitude, time interval, slope, etc. of the BCG based on the R peak of the ECG, thus enabling a BCG signal to be analyzed.

For example, FIG. 2 illustrates an example of the above-described BCG signal analysis, wherein I denotes a signal emitted when the heart ejects blood through the main artery, and J denotes a signal emitted when the blood flows downwards to the lower part of the body, so that the contractile force of the left ventricle may be derived from the magnitudes of the I and J signals.

A basic procedure for the above-described BCG signal analysis is to determine the location of a J peak on a time axis from a BCG waveform that has been obtained. Conventional methods of detecting the peak include thresholding, template matching, wavelet analysis, etc., and among these methods, template matching is the most widely used due to the characteristics of the BCG signal.

However, such template matching is disadvantageous in that when a prepared standard template does not match a BCG signal that has been measured and input, serious errors may be caused in the detection of a peak.

In particular, in order to analyze a BCG signal using template matching in a special environment in which a detected BCG signal is susceptible to changes in the posture or the circumstances of an examinee, as in a vehicle, it is impossible to suitably analyze the BCG signal and to take follow-up actions based on the results of the analysis unless a template is suitably changed and there is a way to cope with such an environment.

That is, in the construction of a system in which a BCG signal is detected and analyzed in a vehicle or the like and then the physical condition of a passenger, such as a driver, is detected and suitable action is taken based on the physical condition, a sensor for detecting a BCG signal must be typically installed under a seat. In this case, when a BCG signal generated from a human body is transferred to the sensor, the covering of the seat, a cushion pad, etc. may influence and change the shape of the BCG signal. Further, since the direction in which the most powerful BCG is generated varies with the direction of the heart relative to the ground, the shape of a signal measured by the sensor may vary depending on the posture of the examinee, such as the forward/backward and leftward/rightward tilts of the upper body of the examinee or the degree of curvature of the upper body. As a result, it is possible to exactly analyze a BCG signal only when the template for analyzing the BCG signal must be able to be suitably changed and used depending on the posture or the like of the examinee.

The information disclosed in this Background section is only for enhancement of understanding of the general background of the invention and should not be taken as an acknowledgement or any form of suggestion that this information forms the prior art already known to a person skilled in the art.

### SUMMARY OF INVENTION

Various aspects of the present invention provide for a ballistocardiogram (BCG) analysis apparatus and method, and a system for utilizing the BCG for a vehicle using the apparatus and method, which can suitably analyze a BCG signal measured from an examinee by suitably taking into consideration a change in the posture or the like of the examinee in a variable environment, such as in a vehicle, thus enabling exact diagnosis of the physical condition of the examinee to be made and suitable follow-up actions to be taken depending on the physical condition.

Various aspects of the present invention provide for a ballistocardiogram (BCG) analysis method, including selecting a basic pattern when measurement of a BCG is initiated; measuring data about BCG for a predetermined period of time; determining whether the basic pattern is suitable for the measured data by comparing the measured data with the basic pattern; if it is determined that the basic pattern is suitable for the measured BCG data, collecting continuous data about the BCG; performing signal processing based on pattern matching using the continuous data; recognizing a biological condition of the examinee using results of the signal processing; and measuring a posture of the examinee, and if the posture of the examinee has changed, searching a pattern database (DB), changing the basic pattern to a suitable pattern, and then performing signal processing.

Various aspects of the present invention provide for a ballistocardiogram (BCG) analysis method, including measuring a posture of an examinee; selecting a pattern depending on the measured posture; processing a signal by performing pattern matching using the selected pattern; and recognizing a biological condition of the examinee using results obtained at the processing the signal.

Various aspects of the present invention provide for a ballistocardiogram (BCG) analysis apparatus, including a posture measurement sensor for measuring a posture of an examinee; a pattern database (DB) constructed by standardizing BCG signals for a variety of individual postures of the examinee and extracting representative patterns; a BCG measurement unit for measuring a BCG signal of the examinee; a signal processing unit for processing the BCG signal measured by the BCG measurement unit so that the BCG signal matches a pattern of the pattern DB, thus calculating desired data; and a biological condition recognition unit for recognizing a biological condition of the examinee using a resulting value obtained by the signal processing unit.

Various aspects of the present invention provide for a system for utilizing a ballistocardiogram (BCG) for a vehicle, including a posture measurement sensor for measuring a posture of an examinee; a pattern database (DB) constructed by standardizing BCG signals for a variety of individual postures of the examinee and extracting representative patterns; a BCG measurement unit for measuring a BCG signal of the examinee; a signal processing unit for processing the BCG signal measured by the BCG measurement unit so that the BCG signal matches a pattern of the pattern DB, thus calculating desired data; a biological condition recognition unit for recognizing a biological condition of the examinee using a resulting value obtained by the signal processing unit; and a condition management unit for driving a device of the vehicle that may influence the biological condition of the examinee depending on the biological condition recognized by the biological condition recognition unit.

The methods and apparatuses of the present invention have other features and advantages which will be apparent from or are set forth in more detail in the accompanying drawings, which are incorporated herein, and the following Detailed Description, which together serve to explain certain principles of the present invention.

### BRIEF DESCRIPTION OF THE DRAWINGS

FIG. 1 is a diagram showing the conventional measurement of a BCG signal.

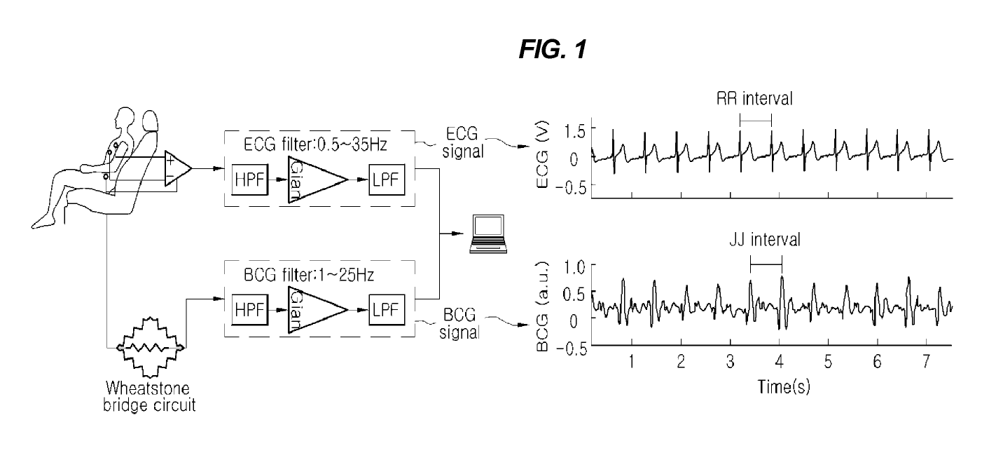

FIG. 2 is a diagram showing an example of the conventional analysis of a BCG signal.

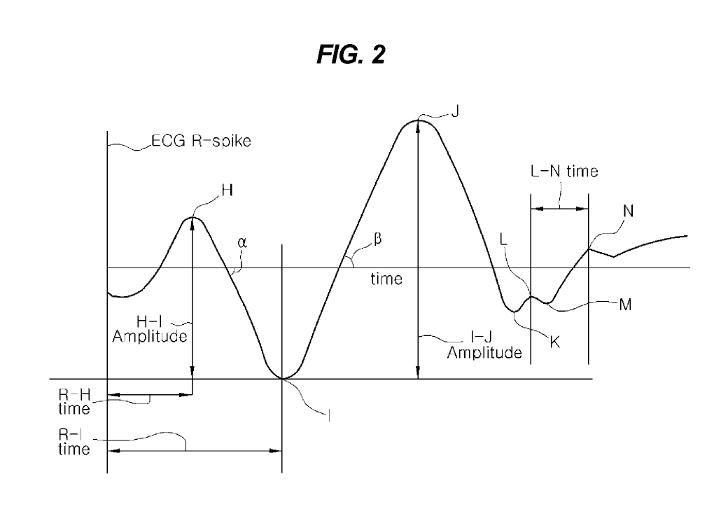

FIG. 3 is a flowchart showing an exemplary BCG analysis method according to the present invention.

FIG. 4 is a block diagram showing an exemplary BCG analysis apparatus according to the present invention.

FIG. 5 is a block diagram showing an exemplary system for utilizing a BCG for a vehicle according to the present invention.

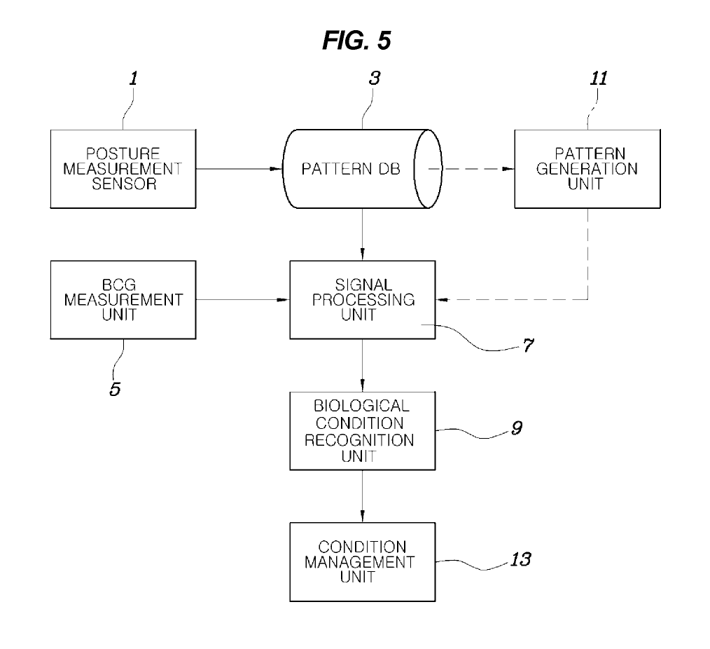

### DETAILED DESCRIPTION

Reference will now be made in detail to various embodiments of the present invention(s), examples of which are illustrated in the accompanying drawings and described below. While the invention(s) will be described in conjunction with exemplary embodiments, it will be understood that present description is not intended to limit the invention(s) to those exemplary embodiments. On the contrary, the invention(s) is/are intended to cover not only the exemplary embodiments, but also various alternatives, modifications, equivalents and other embodiments, which may be included within the spirit and scope of the invention as defined by the appended claims.

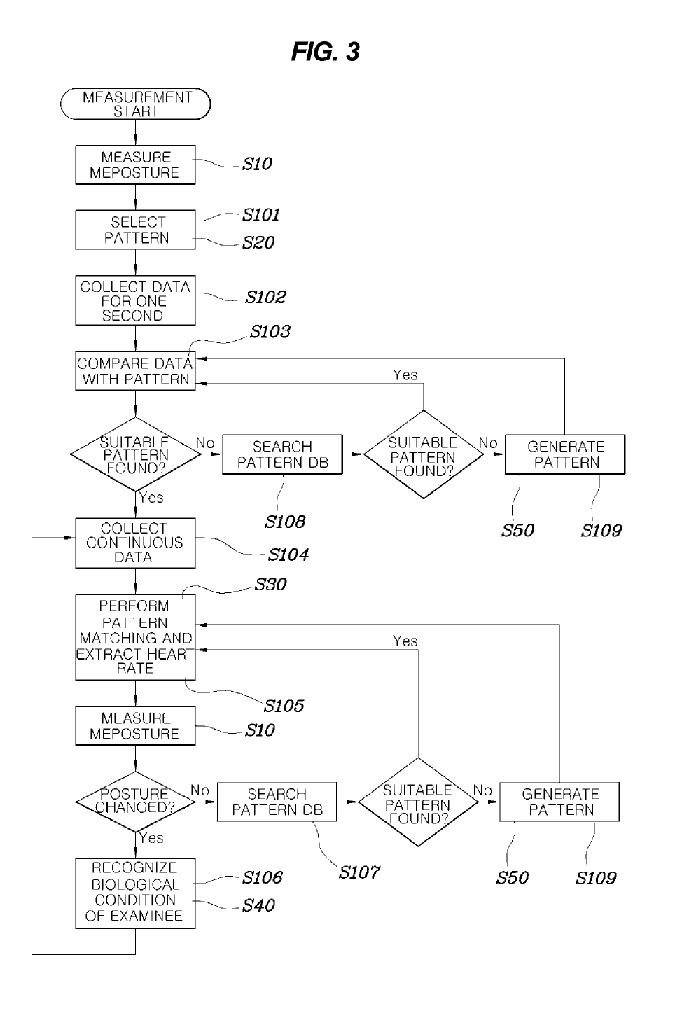

Referring to FIG. 3, an embodiment of a ballistocardiogram (BCG) analysis method according to the present invention includes the posture measurement step S10 of measuring the posture of an examinee; the pattern selection step S20 of selecting a pattern depending on the measured posture; the signal processing step S30 of processing a signal by performing pattern matching using the selected pattern; and the recognition step S40 of recognizing the biological condition of the examinee using the results of the signal processing step S30.

That is, the pattern selection step S20 is performed depending on the posture of the examinee measured at the posture measurement step S10, so that the signal processing step S30 may be performed by always performing suitable pattern matching regardless of the examinee assuming various postures and the posture of the examinee changing, thus enabling more prompt and precise analysis of the BCG to be conducted.

After the posture measurement step S10, the posture change determination step of determining whether the posture of the examinee has changed may be further performed. If it is determined that the posture of the examinee has changed, a new pattern is selected from a pattern database (DB), and then the signal processing step S30 is performed.

In this way, the posture of the examinee is continuously monitored, and the changes in the posture are incorporated in real time, so that it is always possible to exactly and promptly analyze the BCG, and thus the receptivity to and operational precision of various services, such as massaging or ventilation based on the results of the analysis, may be anticipated.

As a result of searching the pattern DB, if it is determined that there is no pattern that matches the measured posture of the examinee, the method may be configured such that the pattern generation step S50 is further performed to generate a pattern most suitable to the current posture of the examinee from patterns corresponding to postures similar to the current posture of the examinee, after which the signal processing step S30 is performed.

That is, since the pattern DB cannot store patterns for all possible postures, if an obscure posture for which it is difficult to determine which specific pattern the measured posture corresponds to has been measured, a new pattern suitable for the measured posture may be generated from patterns corresponding to postures similar to the measured posture, and then the new pattern may be applied to the method.

In detail, the embodiment of FIG. 3 includes the step S101 of, when the measurement of a BCG is initiated, selecting a basic pattern; the step S102 of measuring data about the BCG for a predetermined period of time; the step S103 of determining whether the basic pattern is suitable for the measured data by comparing the measured data with the basic pattern; the step S104 of, if the basic pattern is suitable for the measured data, collecting continuous data about the BCG; the step S105 of performing signal processing based on pattern matching using the continuous data; the step S106 of recognizing the biological condition of the examinee using the results of performing the signal processing; and the step S107 of measuring the posture of the examinee, and if the posture has changed, searching the pattern DB for a suitable pattern, changing the basic pattern to the suitable pattern, and then performing signal processing.

That is, once the measurement of a BCG is initiated, the basic pattern is selected. Then, the BCG data is measured and collected for a predetermined period of time, for one second for example, and is compared with the basic pattern, so that whether the basic pattern is suitable for the BCG data is determined. If it is determined that the basic pattern is suitable for the BCG data, continuous BCG data is collected, and then pattern matching is performed using the pattern and signal processing for extracting other pieces of required information is performed. The biological condition of the examinee, such as the stress level or arousal state of the examinee, may be recognized using the results of the signal processing.

Of course, as the result of determining whether the basic pattern is suitable for the measured data by comparing the measured data with the basic pattern, if the basic pattern is not suitable, the step S108 of searching the pattern DB and then changing the basic pattern to a suitable pattern is performed. If no pattern suitable for the posture of the examinee is found as the result of searching the pattern DB, the step S109 of generating a pattern most suitable for the current posture of the examinee from patterns corresponding to postures similar to the current posture is further performed.

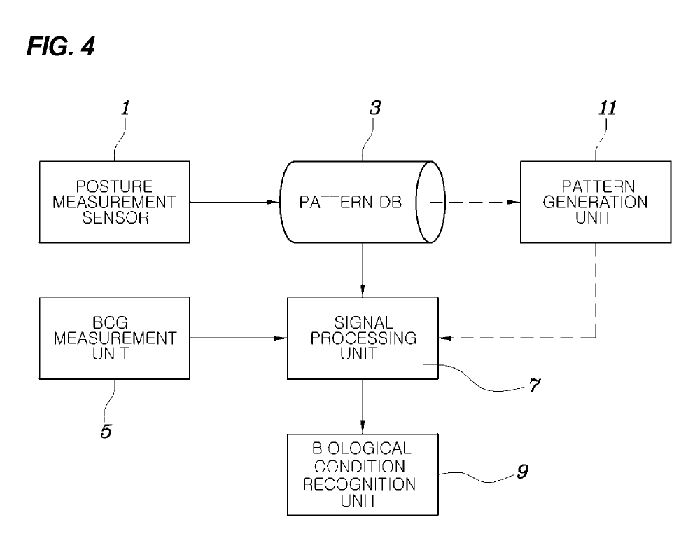

Hereinafter, the construction of a BCG analysis apparatus for implementing the BCG analysis method according to the present invention will be described with reference to FIG. 4. The BCG analysis apparatus includes a posture measurement sensor 1, a pattern DB 3, a BCG measurement unit 5, a signal processing unit 7, and a biological condition recognition unit 9. The posture measurement sensor 1 measures the posture of an examinee, and the pattern DB 3 is constructed by standardizing BCG signals for a variety of individual postures of the examinee and extracting representative patterns. The BCG measurement unit 5 measures the BCG signal of the examinee. The signal processing unit 7 processes the BCG signal measured by the BCG measurement unit 5 so that the BCG signal matches the pattern of the pattern DB, thus calculating the desired data. The biological condition recognition unit 9 recognizes the biological condition of the examinee using a resulting value obtained by the signal processing unit 7.

The BCG analysis apparatus may further include a pattern generation unit 11 for generating a pattern most suitable for the currently measured posture of the examinee from patterns corresponding to postures similar to the current posture of the examinee when no pattern corresponding to the posture measured by the posture measurement sensor 1 is found in the pattern DB.

In this case, the posture measurement sensor 1 may be implemented as a selective combination of a camera for capturing the examinee, an inertial sensor attached to a human body, a tilt sensor for a seat, an elasticity sensor for a seat belt, a pressure sensor embedded in the seat, etc. Using those sensors, the posture measurement sensor 1 detects the movement of the center of gravity of the examinee and determines the forward/backward and leftward/rightward tilts of the examinee or the degree of curvature of the examinee.

Meanwhile, the system for utilizing a BCG for a vehicle may be constructed using the above-described BCG analysis method and apparatus. The system include a posture measurement sensor 1, a pattern DB 3, a BCG measurement unit 5, a signal processing unit 7, a biological condition recognition unit 9, and a condition management unit 13. The posture measurement sensor 1 measures the posture of an examinee, and the pattern DB 3 is constructed by standardizing BCG signals for a variety of individual postures of the examinee and extracting representative patterns. The BCG measurement unit 5 measures the BCG signal of the examinee. The signal processing unit 7 processes the BCG signal measured by the BCG measurement unit 5 so that the BCG signal matches the pattern of the pattern DB, thus calculating the desired data. The biological condition recognition unit 9 recognizes the biological condition of the examinee using a resulting value obtained by the signal processing unit 7. The condition management unit 13 drives the devices of the vehicle that may influence the biological condition of the examinee depending on the biological condition recognized by the biological condition recognition unit 9.

That is, the system is implemented such that the condition management unit 13 is added to the BCG analysis apparatus according to the present invention. The condition management unit 13 may be a display device for displaying the biological condition of the examinee so that the examinee can view the biological condition, a voice warning device for generating voice guidance, a ventilation device for performing ventilation, or a massaging device provided on a seat.

As described above, the present invention is advantageous in that a BCG signal measured from an examinee can be suitably analyzed by suitably taking into consideration a change in the posture or the like of the examinee in a variable environment, such as in a vehicle, so that exact diagnosis of the physical condition of the examinee is possible, so that suitable follow-up actions, such as providing a massaging function to the examinee in a vehicle or the like or ventilating the interior of the vehicle, may be taken based on the physical condition, thus maximizing the marketability of vehicles.

For convenience in explanation and accurate definition in the appended claims, the terms upper or lower, front or rear, inside or outside, and etc. are used to describe features of the exemplary embodiments with reference to the positions of such features as displayed in the figures.

The foregoing descriptions of specific exemplary embodiments of the present invention have been presented for purposes of illustration and description. They are not intended to be exhaustive or to limit the invention to the precise forms disclosed, and obviously many modifications and variations are possible in light of the above teachings. The exemplary embodiments were chosen and described in order to explain certain principles of the invention and their practical application, to thereby enable others skilled in the art to make and utilize various exemplary embodiments of the present invention, as well as various alternatives and modifications thereof. It is intended that the scope of the invention be defined by the Claims appended hereto and their equivalents.

What is claimed is:

1. A ballistocardiogram (BCG) analysis method, comprising:
selecting a basic pattern when measurement of a BCG is initiated;
measuring data about BCG for a predetermined period of time;
determining whether the basic pattern is suitable for the measured data by comparing the measured data with the basic pattern;
if it is determined that the basic pattern is suitable for the measured BCG data, collecting continuous data about the BCG;
performing signal processing based on pattern matching using the continuous data;
recognizing a biological condition of the examinee using results of the signal processing; and
measuring a posture of the examinee, and if the posture of the examinee has changed, searching a pattern database (DB), changing the basic pattern to a suitable pattern, and then performing signal processing.

2. The BCG analysis method according to claim 1, wherein if it is determined that the basic pattern is not suitable as a result of determining whether the basic pattern is suitable for the measured data by comparing the measured data with the basic pattern, the basic pattern is changed to a suitable pattern by searching the pattern DB.

3. The BCG analysis method according to claim 1, further comprising:
if no pattern suitable for the posture of the examinee is found as a result of searching the pattern DB, generating a pattern most suitable for a current posture of the examinee from patterns corresponding to postures similar to the current posture of the examinee.

4. A ballistocardiogram (BCG) analysis method, comprising:
measuring a posture of an examinee;
selecting a pattern depending on the measured posture;
processing a signal by performing pattern matching using the selected pattern; and
recognizing a biological condition of the examinee using results obtained at the processing the signal.

5. The BCG analysis method according to claim 4, further comprising, after the measuring the posture, determining whether the posture of the examinee has changed,
wherein if it is determined that the posture of the examinee has changed, a new pattern is selected from a pattern database (DB), and thereafter the processing the signal is performed.

6. The BCG analysis method according to claim 5, further comprising:
as a result of searching the pattern DB, if it is determined that no pattern that matches the measured posture of the examinee is found in the pattern DB, generating a pattern most suitable for a current posture of the examinee from patterns corresponding to postures similar to the current posture of the examinee,
wherein, after the generating the most suitable pattern, the processing the signal is performed.

7. A ballistocardiogram (BCG) analysis apparatus, comprising:
a posture measurement sensor for measuring a posture of an examinee;
a pattern database (DB) constructed by standardizing BCG signals for a variety of individual postures of the examinee and extracting representative patterns;
a BCG measurement unit for measuring a BCG signal of the examinee;
a signal processing unit for processing the BCG signal measured by the BCG measurement unit so that the BCG signal matches a pattern of the pattern DB, thus calculating desired data; and
a biological condition recognition unit for recognizing a biological condition of the examinee using a resulting value obtained by the signal processing unit.

8. The BCG analysis apparatus according to claim 7, further comprising:
a pattern generation unit for generating a pattern most suitable for a currently measured posture of the examinee from patterns corresponding to postures similar to the currently measured posture of the examinee if no pattern corresponding to the posture measured by the posture measurement sensor is found in the pattern DB.

9. A system for utilizing a ballistocardiogram (BCG) for a vehicle, comprising:
a posture measurement sensor for measuring a posture of an examinee;
a pattern database (DB) constructed by standardizing BCG signals for a variety of individual postures of the examinee and extracting representative patterns;
a BCG measurement unit for measuring a BCG signal of the examinee;
a signal processing unit for processing the BCG signal measured by the BCG measurement unit so that the BCG signal matches a pattern of the pattern DB, thus calculating desired data;
a biological condition recognition unit for recognizing a biological condition of the examinee using a resulting value obtained by the signal processing unit; and
a condition management unit for driving a device of the vehicle that may influence the biological condition of the examinee depending on the biological condition recognized by the biological condition recognition unit.
